(19) United States
(12) Patent Application Publication
(10) Pub. No.: US 2014/0199410 A1
(43) Pub. Date: Jul. 17, 2014

(54) EDIBLE BROWN ALGAE EXTRACT WITH A LOW IODINE CONTENT

(71) Applicant: Guy Potier, Paris (FR)

(72) Inventor: Guy Potier, Paris (FR)

(73) Assignee: ALGUES ET MER, Ile D'Ouessant (FR)

(21) Appl. No.: 14/114,319

(22) PCT Filed: Apr. 23, 2012
(86) PCT No.: PCT/FR12/50887

§ 371 Date: Jan. 13, 2014

(30) Foreign Application Priority Data
Apr. 27, 2011 (FR) 1153581

(51) Int. Cl. A61K 36/03; A61K 33/00; A61K 33/42; A23L 1/30; A61K 31/734; A61K 31/715; A61K 31/05; A61K 33/06; A61K 33/18

(52) U.S. Cl. CPC A61K 36/03; A61K 33/06; A61K 33/00; A61K 33/42; A61K 33/18; A61K 31/734; A61K 31/715; A61K 31/05; A23L 1/3002
USPC 424/601

(57) ABSTRACT

The invention relates to a brown algae extract containing less than 50 ppm of iodine and between 5 and 15%, and preferably between 8 and 10%, of polyphenols, said percentage of polyphenols being expressed as a chlorogenic acid equivalent in relation to the dry weight of the extract, the invention also relating to an oral absorption composition comprising such an extract, and to a method for preparing the extract according to the invention.

### TECHNICAL FIELD

The present invention relates generally speaking to a brown algae extract, notably for food, and its process of production, this extract having a very low iodine content, a high content of polyphenols (or polyphenolic compounds) and significant levels of alginates, polysaccharides and minerals.

The present invention also relates to a composition for oral absorption (intake), containing such an extract.

### BACKGROUND

As a society economically develops, the human being, getting wealthier, looks for, whatever the culture, more comfort (in the meaning of physically stressless life) and a more varied and richer food (including by increased consumption of meat or more sophisticated manufactured goods).

The combination of these two phenomena favors the development of a set of risk factors affecting health. By reducing the physical effort, the human being decreases energy consumption, but, eating in plenty and in a richer way, he consumes far more than its nutritional needs. This leads to a syndrome, referred to as metabolic.

Metabolic syndrome is defined, for the purpose of the present invention, as a set of metabolic disturbances, which although does not fulfill the strict definition of disease, strongly predisposes to the development and progression of true chronic diseases such as hypertension, arteriosclerosis, or diabetes.

For example, a pre-diabetic is six times more likely to develop diabetes than a normal individual.

However, the medical care occurs most often only when the disease is found out. Lifestyle and dietary rules are therefore essential (better balanced meals and maintaining a physical activity) but are insufficient at this stage of the disease, which then makes necessary the use of drugs (e.g., cholesterol-lowering, anti-diabetic . . . ).

Some food products have been specifically developed to limit the development of these diseases.

These include products with polyunsaturated fatty acids or fat adsorbers, but they do not always have a real long-term interest. Indeed, the endogenous part of these diseases is predominant (the influence of food intake on hypercholesteromia is estimated at 20%).

### BRIEF SUMMARY

The purpose of the present invention thus is the manufacturing of food products (extracts and compositions for oral intake containing such extracts) that permit to take care of individuals suffering from metabolic syndrome, and particularly pre-diabetics.

Composition for oral intake is, within the meaning of the present invention, a product obtained by processing a food, that is taken orally, closely to meals, in addition to the everyday diet, this product having only a strictly digestive action (no absorption being necessary to the digestive activity of the product and the effect taking place in the alimentary bolus).

A well-known solution to take care, on a long-term basis and effectively, of individuals with metabolic syndrome or pre-diabetic individuals is the use of food brown algae extracts. Indeed, the inhibitory potency of brown algae extracts, known to be rich in phlorotannins, on some enzymes in the digestive system has already been highlighted in several publications [1] to [3] and few other food plants have such activity.

These enzymes break down complex nutrients in assimilated nutrients (especially sugars) and are therefore an important factor influencing postprandial blood levels. By inhibiting the activity of these enzymes, the nutrients in the alimentary bolus are more slowly digested and absorbed during the meal.

The glycemic, triglyceridic and insulinemic responses are consequently lowered.

The brown algae extracts can reduce the negative impact of excessive food intake compared to needs, while providing some necessary elements for the body such as:
minerals (calcium, potassium, phosphorus, magnesium, iodine . . . ), whose contributions are essential to the body [4];
alginates which form a calcic gel in the stomach, contributing to the enzymatic inhibition activity of the polyphenols (blocking sites of attack of nutrients by enzymes) and generating an effect of satiety [5], and
fucoidans, which inhibit the binding of Helicobacter pylori to digestive walls, limiting the risk of ulcers [6] or limiting the enzymatic hydrolysis of starch thanks to inhibiting effects [7].

The well-known brown algae extracts are though rich in phlorotannins, and therefore have an inhibitory activity on some enzymes in the digestive system. But they have an iodine content remaining high, even when the extracts are treated for deiodination. Indeed, during this deiodination, iodine is only partially removed and the residual content (300 ppm for the less rich ones) limits the maximum daily intake (500 mg), and therefore the clinical relevance of the product.

It is therefore necessary that the iodine content of the extract is minimized to allow the use of the extract in effective doses.

Among the plant extracts currently marketed, may be mentioned the extract marketed by the applicant company itself under the brand InSea2® (which is a brown algae extract acting as an inhibitor of enzymes digesting carbohydrates, the alpha-amylase and alpha-glucosidase), the brown algae extract sold by Bioserae under the Id-Alg™ brand, the white kidney bean extract marketed by Ingredia Nutritional under Brand Phase2®, or the brown algae extract manufactured by the company Diana Naturals Company, and being the object of the French patent application FR2914190. However, the latter product being designed to fight against inflammation, arthritis and allergies, it is away from the field of the present invention.

One may also include pharmaceutical active ingredients Acarbose INN (marketed by Bayer under the brand GLUCOR®™) and Orlistat INN (marketed by Roche Laboratories and Glaxo SmithKline under the brands Xenical®™ and Alli™ respectively).

However, the pharmaceutical active ingredient Acarbose INN has the disadvantage of causing flatulence, and thus the non-observance of treatment, while the pharmaceutical active ingredient Orlistat INN has the disadvantage of blocking fat absorption, and thus cause strong acceleration of transit, and thus ultimately non-compliance of the treatment by the patient.

As regards algae extract, the methods used to remove iodine during manufacturing are not selective: not only do they require to go through a chemical step of removing colloids (acidification for most, causing structural changes of the extract), but they also remove other compounds, essential for the body, such as minerals.

The main disadvantages of these methods are listed below:
during extraction at acid pH (method described in FR2914190), too long duration, low pH and high temperature cause hydrolysis of polyphenols, so a loss of efficiency and a significant change in “native” molecules;
there is also a risk of presence of residual solvent in the extracts depending on the nature of the solvents used during the extraction (ethanol, other organic solvents);
to prevent any clogging during a phase of iodine removal by tangential membrane filtration, alginates are necessarily removed, either by acidification (production of the product InSea2®), or precipitation, with calcium chloride for example (process described in FR2914190), these additional steps not only lead to the removal of alginates (biological interest), but also to additional costs;
elimination of iodine with tangential membrane filtration is realized by diafiltration (FR2914190): part of the iodine is driven in the permeate, but also some of the minerals and dry matter; the more you want to eliminate iodine, the more you must diafilter, and the more the extract is low in molecules the molecular weight of which is less than the cutoff point (InSea2®: tangential ultrafiltration). In addition, some contaminants present in the native extract and whose molecular weight is higher than the cutoff point of the membrane will be concentrated in the retentate;
during removal of iodine by chromatography (FR2914190) with adsorbent resin, the use of organic solvent(s) is required in order to elute the polyphenols.

A more or less minimal quantity of solvent(s) will persist after the phase of concentration, which can cause some problems during the drying of the extract.

### DETAILED DESCRIPTION AND EXAMPLES

The purpose of the present invention is therefore the development of a brown algae extract with a reduced iodine content, while maintaining the original level of minerals, alginates, fucoidans and phlorotannins (polyphenols), in order to be as close as possible to the native extract.

More particularly, the present invention purpose is an algae extract, characterized in that it contains less than 50 ppm iodine and between 5 and 15%, and preferably between 8 and 10%, polyphenols, said percentage of polyphenols being expressed as chlorogenic acid equivalent on a dry matter of the extract basis.

Polyphenols mean, for the purpose of the present invention, the phlorotannins (or phenolic antioxidants) generally present in brown algae: these are chemical structures that are widely found in land plants and algae. The particularity of algae polyphenols is that they are very large molecules, of which predominant monomer is phloroglucinol (hence the name of phlorotannins).

The algae extract according to the invention has an inhibitory activity on alpha-amylase, alpha-glucosidase and lipase.

Reduced iodine content of algae extract according to the invention allows a daily dose up to 3 g of extract according to the invention, thus complying with the daily intake of iodine recommended by the European Union in Regulation 2006/352 establishing the maximum daily amount of iodine at 150 μg/day (see also in this regard the relevant French regulations as published in JORF 123 (Journal Officiel de la République Francaise) dated 9 May 2006).

The algae extract according to the invention can advantageously be obtained from brown algae, preferably from alimentary ones.

The algae extract according to the invention can advantageously be obtained from algae (which can be in particular microalgae or macroalgae) belonging to at least one species selected from the species Ascophyllum sp., Fucus sp., Himanthalia sp., Undaria sp. And Laminaria sp.

For the present invention, algaes of the species Ascophyllum nodosum are preferably used.

Advantageously, the algae extract according to the invention may contain between 5 and 9% of alginates, percentage expressed as glucuronic acid equivalent compared to dry matter of the extract. The presence of alginates in the algae extract according to the invention enables the forming of a calcic gel that contributes to the enzymatic inhibition done by phlorotannins and induces a satiety effect. In addition, the presence of alginates, also commonly used for the treatment of gastroesophageal reflux, would, thanks to their chelating power, detoxify the body.

The algae extract according to the invention may also advantageously contain the majority of minerals (except iodine) contained in said algae prior to processing, especially calcium, magnesium, potassium and phosphorus.

The presence of these minerals is essential to the proper functioning of the human body (multiple actions).

Finally, the algae extract according to the invention may also advantageously contain fucoidans, of which properties are numerous and in many areas (gastric ulcer prevention, relief of stomach disorders, etc.). In particular, fucoidans present inhibitory effects on the enzymatic hydrolysis of starch.

The present invention also relates to a composition for oral intake comprising an algae extract according to the invention.

According to a first embodiment for producing the composition for oral intake according to the invention, it is free from carrier, the algae extract being incorporated in said composition without any addition.

According to a second embodiment for producing the composition for oral intake according to the invention, it comprises an orally acceptable carrier, present in said composition in an amount of 0.5% to 5% in weight relative to the total weight of said composition.

As examples of carriers used in the compositions according to the invention, thinners and lubricants used for setting dosage form of the extract, especially magnesium stearate, can be mentioned.

The compositions according to the invention may be in any oral galenic form, including capsules, tablets, pills, phials, capsules, herb teas, solutions or oral suspensions, powders, flakes or granules.

The present invention also relates to a process for preparing an algae extract according to the invention, with following steps:
solid/liquid extraction of algae, dried or not, grinded or not is performed;
separation of the liquid phase and the solid phase of the mixture is performed;
purification of liquid phase is performed, to obtain a purified aqueous extract;
drying of the obtained aqueous extract is performed;
   said process is characterized in that it comprises, between the step of liquid phase purification and the step of drying the said aqueous extract, a step of specific deiodination of said purified aqueous extract, which is carried out by going through an ion-exchange resin.

The process according to the invention permits, without using an organic solvent or removing colloids such as alginates, to obtain a brown algae extract with a decrease in iodine content (equal to or less than 50 ppm), which is greater than a method of prior art, while having a standardized polyphenol content.

In addition, the extract obtained is closer to the native extract (no loss in alginates or fucoidans, no loss in inhibitory activity, little loss of essential minerals such as calcium, potassium, magnesium and phosphorus).

In the method according to the invention, the drying step can advantageously be carried through spray-drying, freeze-drying, vacuum oven or fluidized bed. Preferably, the drying is performed by spray-drying.

In a particularly advantageous embodiment for producing the process according to the invention, the drying step is preceded by a step of concentration and/or standardization of said aqueous extract by addition of soluble dietary fiber (including wheat, corn, etc.) to obtain a polyphenol content between 5 and 15%, and preferably between 8 and 10%.

By standardization, it is meant, in the sense of the present invention, as a step to adjust the polyphenol content in the extract, whatever are the nature or origin of the algae used for obtaining the extract.

As examples of soluble dietary fiber that could be used in the framework of the present invention, the soluble dietary fiber marketed by Roquette under NUTRIOSE® brand may be mentioned.

The present invention also relates to the use of the composition according to the invention for weight management, metabolic syndrome management, glycemic control (prediabetes), limiting the overweight, lipid control (high or subnormal cholesterol level), minor dyspeptic disorders, digestive acidity or mineral supplementation in humans or animals.

For these different uses, the disease is not yet declared.

Finally, the present invention also relates to a composition for oral intake containing an algae extract according to the invention, for use as a medicine.

More specifically, such a composition according to the invention can be used as a medicine in the treatment of diabetes, hypercholesterolemia, hypertension, cerebrovascular accident (stroke), cardiovascular disease, obesity, dyspepsia (digestive disorders), gastroesophageal reflux disease (GERD), stomach ulcers and mineral deficiencies for human or animals.

The invention is illustrated by the following examples, which are not exhaustive.

### EXAMPLES

### Example 1

### Preparation of a Brown Algae Extract According to the Present Invention

Brown algae Ascophyllum nodosum are harvested; fresh or dried, they are ground.

This is followed by aqueous maceration, of which main objective is to extract the main components of the alga, namely polyphenols, fucoidans, alginates and minerals.

The extraction temperature is between 40° C. and 95° C., and the extraction lasts at least six hours.

Then, to exhaust the solid residue and recover the aqueous extract, solid-liquid separation is carried out on a vibrating sieve or centrifugal separator.

This is followed by a clarification of the so obtained aqueous extract, in order to remove a maximum of microparticles in suspension and thus avoid fouling the resin at the next step, this operation being carried out on a filter press, plate filter or decanter centrifuge.

This is followed by a step of removing the iodine out of extract to reach a final content less than 50 ppm. The removing of iodine from the aqueous extract is made by passing through an ion exchange resin at room temperature (between 15° C. and 25° C.).

The extract is then concentrated under vacuum at low temperature to reach between 10 and 15% of dry matter, then it is standardized by addition of soluble dietary fiber to obtain a final polyphenol content between 8 and 10%.

The liquid extract thus obtained is then spray-dried to obtain a brown and fine powder, rich in polyphenols.

### Example 2

### Assay of Iodine, Polyphenols, Fucoidans and Alginates in the Extract of Example 1

For iodine, the assay is performed by titration with sodium thiosulfate (results in ppm) with the method from Codex on Nutrition and dietary food, ESPA/CN 109/84.

For polyphenols, this assay is performed according to the Folin method and content is expressed as chlorogenic acid.

For Alginates, this assay is performed according to the BLUMENKRANTZ and G.A-HANSEN method, with metahydroxydiphenyl, and content is expressed as glucuronic acid.

For total sugars, assay is performed according to the DUBOIS method, of which the principle is to determine the glucose content.

The results of these assays are given below:
alginates content: 5 to 9% based on the dry matter,
polyphenols content, 8 to 10% expressed as glucuronic acid equivalent;
total sugar content: around 30%;
iodine content: less than 50 ppm.

### Example 3

### In Vitro Evaluation of the Inhibitory Activity of Digestive Enzymes by the Extract of Example 1

We evaluate in vitro inhibitory activity of digestive enzymes by the algae extract of Example 1, obtained by the process according to the invention, but from two different production batches.

The extract obtained in Example 1 has the following characteristics:
inhibitory activity of alpha-amylase at 1 g/L: >80%;
inhibitory activity of alpha-glucosidase at 0.1 g/L: >80%;
inhibitory activity of the lipase at 0.1 g/L: >10%.

The methods for determining enzyme activities are conventional colorimetric methods.

The principle is as follows:
starch is incubated with an enzyme (for example human salivary amylase). This incubation results in the digestion of starch and maltose production,
a colored product is added and is intended to stop the enzymatic reaction and to reveal the presence of maltose;
a colorimetric assay to quantify, at a precise wavelength, the residual maltose,
adding an “inhibitor” to the solution before incubation leads to maltose decrease and therefore to its colorimetric assay.
comparisons between control solution and test solution permit to calculate a percentage of inhibition.

These results are detailed in Table 1 below, which also shows the inhibitory activities of a brown algae extract known in the prior art, InSea2® on one hand, and those of the pharmaceutical active ingredient Acarbose INN on the other hand (in this example, we used the one sold by Bayer under brand name GLUCOR™).

TABLE 1
Comparison of the inhibitions of the extract according
to the invention with an extract of the prior art
and a pharmaceutical active ingredient

| | | Extract | Extract | | |
|---|---|---|---|---|---|
| | | according | according | | |
| | Concen- | to the | to the | | |
| Inhibitory | tration | invention | invention | | |
| Activity | in g/l | (batch N° 1) | (batch N° 2) | InSea2 ® | Glucor ™ |
| Alpha- | 1.0 | 80 | 87 | 83 | 93 |
| amylase | | | | | |
| Alpha- | 0.1 | 92 | 89 | 99 | 100 |
| glucosi- | | | | | |
| dase | | | | | |
| Lipase | 1.0 | 29 | 36 | — | — |

Table 1 shows that for results of the inhibitory level of digestive enzymes, the in vitro results obtained for algae extracts according to the invention (regardless which batch it comes from) are equivalent to those obtained with the algae extract of the prior art InSea2® or with pharmaceutical active ingredient GLUCOR®™.

1. Algae extract, comprising less than 50 ppm of iodine and between 5 and 15% of polyphenols, said percentage of polyphenols being expressed in chlorogenic acid equivalent compared with the dry matter of the extract.

2. Algae extract according to claim 1, comprising between 8 and 10% of polyphenols, said percentage of polyphenols being expressed in chlorogenic acid equivalent compared with the dry matter of the extract.

3. Algae extract according to claim 1, wherein said algae are brown.

4. Algae extract according to claim 1, wherein said algae are food algaes.

5. Algae extract according to claim 1, comprising between 5 and 9% of alginates, said percentage of alginates being expressed in glucuronic acid equivalent compared with the dry matter of the extract.

6. Algae extract according to claim 1, comprising the majority of minerals content, except iodine, present in said algae before their processing by extraction.

7. Algae extract according to claim 1, wherein said algae belongs to at least one of the following species: Ascophyllum sp., Fucus sp., Himanthalia sp., Undaria sp., and Laminaria sp.

8. Composition for oral intake comprising an algae extract such as defined according to claim 1.

9. Composition for oral intake according to claim 8, wherein the composition is free from any carrier.

10. Composition for oral intake according to claim 8, comprising an acceptable carrier for oral intake, present in said extract in a content of 0.5% to 5 wt % compared with the total weight of said composition.

11. Composition according to claim 10, wherein the carrier is magnesium stearate.

12. Composition according to claim 8, wherein the composition is in the form of capsule, pastille, tablet, phial, capsule, herb tea, solution or drinkable suspension, powder, flake, or granule.

13. Composition according to claim 8, wherein the composition is a food supplement.

14. Composition according to claim 8, wherein the composition is food.

15. Process for the preparation of an algae extract such as defined according to claim 1, including the following stages:
solid/liquid extraction of the algae, dried or not, ground or not before is performed;
separation of liquid phase and solid phase of the mixture is performed;
purification of liquid phase is performed, to obtain a purified aqueous extract;
recovery of the aqueous extract, which is then dried is performed;
further comprising, between the stage of purification of the liquid phase and the stage of drying of said aqueous extract, a stage of specific deiodination of said purified aqueous extract which is done by going through an ion-exchange resin.

16. Process according to claim 15, wherein the drying is done by spray-drying, freeze-drying, vacuum oven or fluidized bed.

17. Process according to claim 15, wherein the drying is preceded by a step of concentration and/or standardization of said aqueous extract through addition of soluble food fibers to obtain a polyphenols content between 8 and 10%.

18. Composition according to claim 8, wherein the composition is configured for weight management, metabolic syndrome, glycemic regulation, limitation of excess weight, lipid control, minor dyspeptic disorders, digestive acidity or minerals supplementation for human beings or animals.

19. Composition for oral intake comprising an algae extract as defined according to claim 1 wherein the algae extract is configured for use as medicine.

20. Composition according to claim 19, wherein the composition is configured as medicine in the treatment of diabetes, hypercholesterolemia, arterial hypertension, cerebrovascular accident (CVA), cardiovascular diseases, obesity, stomach dyspepsia, gastroesophageal reflux disease (GERD), stomach ulcers and mineral deficiencies of human beings or animals.
